(19) United States
(12) Patent Application Publication
(10) Pub. No.: US 2025/0067707 A1
(43) Pub. Date: Feb. 27, 2025

(54) CROSS-GAP-NANOPORE HETEROSTRUCTURE DEVICE AND METHOD FOR IDENTIFYING CHEMICAL SUBSTANCE

(71) Applicant: University of Kentucky Research Foundation, Lexington, KY (US)

(72) Inventor: Douglas R. Strachan, Lexington, KY (US)

(21) Appl. No.: 18/943,349

(22) Filed: Nov. 11, 2024

Related U.S. Application Data
(63) Continuation-in-part of application No. 16/901,393, filed on Jun. 15, 2020.
(60) Provisional application No. 62/861,090, filed on Jun. 13, 2019.
(60) Provisional application No. 63/037,689, filed on Jun. 11, 2020.

(51) Int. Cl. G01N 27/447; B03C 5/00; C12Q 1/6869; G01N 33/487

(52) U.S. Cl. CPC G01N 27/44791; B03C 5/005; C12Q 1/6869; G01N 27/4473; G01N 33/48721

(57) ABSTRACT

A heterostructure device and method allow for detection and identification of a chemical substance. The device includes one or more atomically-thin conducting layers and one or more atomically-thin insulating layers including one or more nanogaps that cross and form one or more nanopores.

### RELATED APPLICATION

This application is a continuation-in-part (CIP) of U.S. patent application Ser. No. 16/901,393, filed on Jun. 15, 2020, which claims priority to U.S. Provisional Patent Application Ser. No. 62/861,090, filed on Jun. 13, 2019, and U.S. Provisional Patent Application Ser. No. 63/037,689, filed on Jun. 11, 2020, the full disclosures of which are incorporated herein by reference.

### GOVERNMENT SUPPORT

This invention was made with government support under Grant No. 1603152 awarded by the National Science Foundation (NSF) through Chemical, Bioengineering, Environmental, and Transport Systems (CBET). The government has certain rights in the invention.

### TECHNICAL FIELD

This document relates generally to devices and methods for determining a chemical substance and, more particularly, to cross-gap-nanopore devices and related methods for determining a chemical substance such as the nucleotide sequence of a strand of ribonucleic acid (RNA) or deoxyribonucleic acid (DNA).

### BACKGROUND

Nanopores consisting of integrated electrodes at the location of the pore could be of tremendous technological use in filtering biological and chemical substances from solutions and in sensing and sequencing molecules, like DNA and RNA, as they translocate through the pore. Such sequencing would have use in the rapid sequencing for human genome and infectious diseases with small test samples. Due to the relatively short timescales for mutations of infections, such as RNA viruses, being able to quickly and efficiently track their genetic variations and progressions within society is extremely important for treating and understanding such diseases.

However, to achieve such capabilities has been hindered due to the fact that it is extremely difficult to accurately and reliably determine genetic sequencing with high throughput using minimal amounts of targeted genetic molecules (i.e., DNA or RNA). High throughput methods in real-time with electrical detection through nanopore translocation could enable this accurate and reliable genetic sequencing of individual genetic molecules. However, current technologies are not yet capable of obtaining the required precise placement of multiple electrodes, having sufficient electrical isolation from each other, at the location of a nanopore.

Advantageously, the cross-gap-nanopore heterostructure device disclosed herein allows precise placement of multiple electrodes at the location of a nanopore while having sufficient electrical isolation from each other to function in an efficient and effective manner.

### SUMMARY

In accordance with the purposes and benefits described herein, a new and improved heterostructure device is provided for identifying or determining a chemical substance including polymers, proteins, genetic material such as strands of DNA and RNA, individual molecules, particles and the like. That heterostructure device comprises; (a) a first conducting layer including a first nanogap, (b) a first insulating layer including a second nanogap and (c) a first nanopore formed at a first crossing point of the first nanogap and the second nanogap. The first nanopore extends through the first conducting layer and the first insulating layer. In one or more particularly useful embodiments, the first nanogap forms a first electrode pair.

In one or more embodiments of the heterostructure device, the first conducting layer is atomically-thin. In one or more embodiments of the heterostructure device, the first insulating layer is atomically-thin. In one or more embodiments of the heterostructure device, both the first conducting layer and the first insulating layer are atomically-thin.

For purposes of this document, the terminology “atomically-thin” means having a thickness of about 1 nm or less.

In one or more possible embodiments of the heterostructure, the heterostructure includes a second atomically-thin conducting layer wherein the first atomically-thin insulating layer is sandwiched between the first atomically-thin conducting layer and the second atomically-thin conducting layer. The second atomically-thin conducting layer includes a third nanogap that crosses the first nanogap and the second nanogap at the first crossing point so that the first nanopore also extends through the second atomically-thin conducting layer.

In one or more possible embodiments of the heterostructure, the first nanogap forms a first electrode pair within the first nanopore and the second nanogap forms a second electrode pair within the first nanopore.

In one or more possible embodiments of the heterostructure, the first conducting layer and the second conducting layer include additional nanogaps that cross the second nanogap in the atomically-thin insulating layer at a second crossing point forming a second nanopore.

In one or more possible embodiments of the heterostructure, additional nanogaps in the first conducting layer and the second conducting layer form additional electrode pairs within the second nanopore.

In one or more possible embodiments of the heterostructure, the heterostructure further includes alternating additional atomically-thin conducting layers and additional atomically-thin insulating layers providing (a) additional electrode pairs in the first nanopore and the second nanopore, (b) additional nanopores at additional crossing points or (c) additional electrode pairs in the first nanopore and the second nanopore and additional nanopores at additional crossing points.

In one or more possible embodiments of the heterostructure, the first atomically-thin conducting layer, the second atomically-thin conducting layer and the additional atomically-thin conducting layers are made from a material selected from a group consisting of graphene, transition metal dichalcogenides (TMDs), borophene, germanene, silicene, stanene, plumbene, phosphorene, antimonene, Si₂BN, borocarbonitrides and combinations thereof.

In one or more possible embodiments of the heterostructure, the first atomically-thin insulating layer and the additional atomically-thin insulating layers are made from a material selected from a group consisting of hexagonal boron nitride, transition metal dichalcogenides (TMDs), bismuthene, borocarbonitrides and combinations thereof.

In accordance with yet another aspect, a cross-gap-nanopore heterostructure is provided. That cross-gap-nanopore heterostructure is adapted for the real-time determination of nucleotide sequencing of a strand of genetic material. In one or more of the many possible embodiments, that genetic material comprises RNA. In one or more of the many possible embodiments, that genetic material comprises DNA. In one or more of the many possible embodiments, that genetic material is a combination of RNA and DNA.

In one or more of the many possible embodiments of the cross-gap-nanopore heterostructure, the cross-gap-nanopore heterostructure may include (a) a plurality of alternating atomically-thin conducting layers and insulating layers and (b) at least one nanopore having stacked electrode pairs. In one or more of the many possible embodiments of the cross-gap-nanopore heterostructure, the cross-gap-nanopore heterostructure may include (a) a plurality of alternating atomically-thin conducting layers and insulating layers and (b) a plurality of nanopores wherein each nanopore of the plurality of nanopores includes at least one individually addressable electrode pair. The cross-gap-nanopore structure may be supported on any suitable porous substrate. In at least one particularly useful embodiment, the nanopores are etched and have a width of 10 nm or less, a length of greater than 100 nm and a depth extending completely through the layer of material in which the nanopore is made. Unlike drilled nanopores, etched nanopores have edges that are crystallographically-ordered and parallel on an atomic scale.

In accordance with yet another aspect, a method of determining a chemical species is also provided. That method includes the step of passing the chemical species through at least one nanopore in a cross-gap-nanopore heterostructure.

The method may also include additional steps including, but not necessarily limited to:
(a) performing lateral electrical detection of the chemical substance as the chemical substance passes through the at least one nanopore;
(b) applying a voltage difference to a first electrode pair in the at least one nanopore;
(c) applying voltage differences to a plurality of different electrode pairs in the at least one nanopore;
(d) simultaneously electrically probing the chemical substance with the plurality of different electrode pairs in the at least one nanopore as the chemical substance passes through the at least one nanopore;
(e) influencing the flow of the chemical substance through the at least one nanopore through application of dielectrophoretic forces; and
(f) combinations of (a)-(e).

In the following description, there are shown and described several embodiments of the new and improved (a) heterostructure devices, (b) cross-gap-nanopore heterostructures, and (c) methods of determining a chemical substance. As it should be realized, the heterostructure devices, cross-gap-nanopore heterostructures and methods are capable of other, different embodiments and their several details are capable of modification in various, obvious aspects all without departing from the heterostructure devices and methods as set forth and described in the following claims. Accordingly, the drawings and descriptions should be regarded as illustrative in nature and not as restrictive.

### BRIEF DESCRIPTION OF THE DRAWING FIGURES

The accompanying drawing figures incorporated herein and forming a part of the specification, illustrate several aspects of the new and improved heterostructure devices, cross-gap-nanopore heterostructures and related methods for determining a sequence of a nucleotide strand and together with the description serve to explain certain principles thereof.

FIG. 1 is a schematic view illustrating one atomically-thin etched insulating layer and one atomically-thin etched conducting layer used to form a single cross-gap-nanopore heterostructure, of which an integrated electrode pair could embody one of the two etched materials.

FIG. 2 illustrates the layers of FIG. 1 stacked together resulting in an individual nanopore of which could have an electrode pair.

FIG. 3 a detailed schematic cross sectional view of a strand of nucleic acid passing through the nanopore of FIG. 2.

FIG. 4 schematically illustrates the heterostructure device of FIG. 3 supported over a pore in a support substrate with electrical contacts of the electrode pair connected to a voltage source and an ammeter.

FIG. 5 schematically illustrates three atomically-thin etched conducting and insulating layers used to form an integrated electrode cross-gap-nanopore heterostructure.

FIG. 6 illustrates the layers of FIG. 5 stacked together resulting in a series of individual nanopores each having a unique combination of electrodes.

FIG. 7 is a detailed schematic top plan view of one nanopore of the plurality of nanopores illustrated in FIG. 6.

FIG. 8 is a detailed schematic cross sectional view of a cross-gap-heterostructure including multiple nanopores.

FIG. 9 illustrates yet another possible embodiment of the cross-gap-nanostructure device including a larger number of alternating atomically-thin etched conducting layers and atomically-thin etched insulating layers.

FIGS. 10A-10C schematically illustrate a two-dimensional crystal (10A) and a nanogap made from such a material (10B and 10C) with edges that are crystallographically-ordered and parallel on an atomic scale.

Reference will now be made in detail to the present preferred embodiments of the cross-gap-nanopore heterostructure devices, heterostructures and related methods, examples of which are illustrated in the accompanying drawing figures.

### DETAILED DESCRIPTION

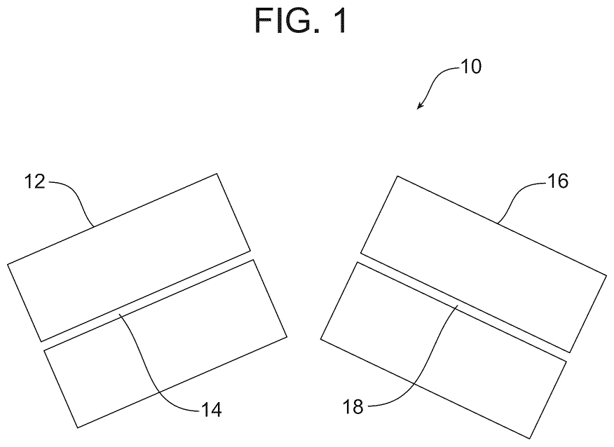

Reference is now made to FIGS. 1-3 which illustrate a first embodiment of a cross-gap-nanopore heterostructure device 10. That device 10 includes (a) a first atomically-thin conducting layer 12 having a first nanogap 14 and (b) a first atomically-thin insulating layer 16 having a second nanogap 18.

Each nanogap 14, 16 may be made by etching and includes edges that are crystallographically-ordered and parallel on an atomic scale. For purposes of this document, “Crystallographically-ordered” nanogap edges is defined the same way that a crystal order is defined. Crystal order is defined based on translational invariance. The strict theoretical definition of crystallographic order is often referred to as a Bravais lattice. On page 64 of Ashcroft and Mermin, the definition of a Bravais lattice is given:

“A Bravais lattice is an infinite array of discrete points with an arrangement and orientation that appears exactly the same, from whichever of the points the array is viewed.”

The criterion of ‘exactly the same’ is a strict definition of crystallographic, allowing for no deviation. Any departure from ‘exactly the same’ involves some sort of defect in the crystal order. Since no crystal is actually infinite in extent, in a practical sense the definition of crystallographic order is taken to be over a physically relevant size-scale such that the local properties are not altered over the physical extent of the crystal. For crystals smaller than this size, changes to the local properties can be observed, an effect referred to as a ‘size effect’. For electronic systems (like graphene electrodes) relevant size effects can be due to electrostatic couplings from interfaces (typically 10s of nanometers in extent), tunneling lengths (under 10 nm), distances to the polymer molecules of interest (˜10 nm or less), or confinements effects of electrons along the edges of the electrodes (again 10s of nanometers in extent). Thus, the lengths of the electrode edges should be long enough to avoid any of these potential size effects.

There are two important aspects of a Bravais lattice that are important in understanding parallel crystallographic edges.

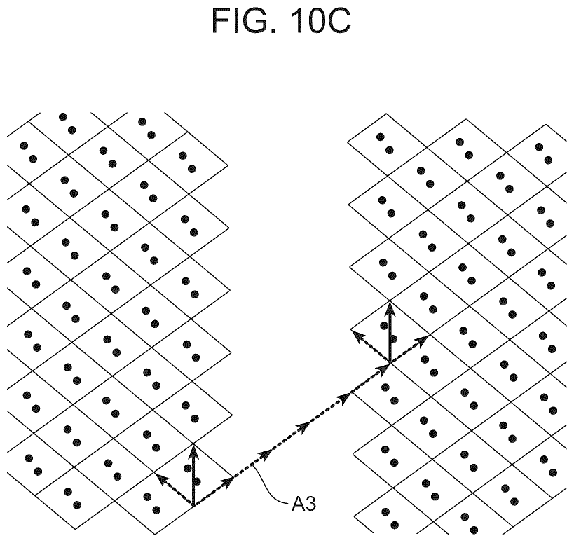

(1) A Bravais lattice can have some sub-structure, known as a ‘basis’, which can contain a plurality of atoms (or molecules) that repeat themselves for every translational unit of the Bravais lattice. We have shown this in FIG. 10A for a two-dimensional lattice consisting of reproduced identical parallelograms, known as unit cells, that each contain a two-atom basis. The entire crystal can be constructed by copying the primitive unit cell and displacing these copies integer multiples of two appropriate primitive lattice vectors (with one possible set in FIG. 10A denoted by the two dotted arrows A1).

(2) A Bravais lattice can exist in various dimensions. The surface of a 3-dimensional (3D) crystal can form a 2-dimensional (2D) Bravais lattice having the same stringent requirements for the 3D case—that there is a repeating unit cell having the exact same arrangement and orientation for all unit cells on the terminating surface. Likewise, and most relevant to the case of crystallographically-ordered nanogap edges, the edges of a 2D crystal can form a one-dimensional (1D) crystal. This is illustrated in FIG. 10B whereby a straight (1D) edge is now terminating two separate 2D crystals. For an edge to be crystallographic, cach unit cell of the edge must have an arrangement and orientation within the physically relevant size scale that is exactly the same as every other unit cell along that edge and within this physically relevant size scale.

Focusing on the left 2D material of FIG. 10B, this means that all possible displacements along the crystallographic edge must reproduce the periodicity of the primitive lattice vectors parallel to a translation vector (the arrow A2 in FIG. 10B that is the sum of the two dotted arrows A1 in FIG. 10A) taking one from one cell to an adjacent cell along the edge. For this reason, a crystallographic edge must be atomically-straight without deviation. For the case of quantum tunneling sequencing devices, the distances between two edges of adjacent electrodes must be controlled on the atomic-scale. Commensurate electrodes, where nanaogaps are formed by the catalytic etching of graphene have this precision. In this case, the etching (or removal) of atoms leaves a gap. Also, in this case, the etching (or removal) of atoms leaves a gap, but the allowed translation vectors on one side of the gap will take one to identical equivalent locations onto the other side of the gap as illustrated by arrow A3 in FIG. 10C. If each edge is to contain lattice sites each having the same exact orientation and arrangement that appears exactly the same (according to the strict definition of a crystal), then both edges must be defined by the same lattice vectors and thus be exactly parallel, again as illustrated in FIG. 10C.

“Parallel on an atomic scale” further refers to case when a 1D edge is formed from a 2D crystal that has unit cells defined by a suitable pair of lattice vectors (A1). A line parallel to a crystallographic 1D edge must be parallel to one made by adding integer multiples of this pair of Bravais lattice vectors for the 2D crystal, otherwise the 1D edge is not crystallographic. For two electrodes to be made of the same underlying 2D material and to both be crystallographic in the same space, requires that they be exactly parallel, with no deviation on the atomic-scale (that is, no atoms out of place), as illustrated in FIG. 10C.

The first atomically-thin conducting layer 12 may be made from any appropriate material suitable for use as an atomically-thin conducting layer including, but not necessarily limited to graphene, transition metal dichalcogenides (TMDs), borophene, germanene, silicene, stanene, plumbene, phosphorene, antimonene, Si₂BN, borocarbonitrides and combinations thereof. The conducting layer 12 may have a thickness of about 1 nm or less.

The first atomically-thin insulating layer 16 may be made from any appropriate material suitable for use as an atomically-thin insulating layer including, but not necessarily limited to hexagonal boron nitride, transition metal dichalcogenides (TMDs), bismuthene, borocarbonitrides and combinations thereof. The insulating layer 16 may have a thickness of about 0.3 nm to 5 nm. For certain applications, either or both the conducting layer 12 and the insulating layer 16 may be much thicker and even have a thickness up to 100 nm or even a micron. Where both the conducting layer(s) 12 and the insulating layer(s) 16 are atomically thin, it is possible to detect individual nucleotides in a DNA or RNA molecule. Here it should also be appreciated that the cross-gap-nanopore heterostructures may comprise substantially any layered combination of conducting, semiconducting and insulating layers with the same or varying thicknesses from atomically thin up to and including one micron.

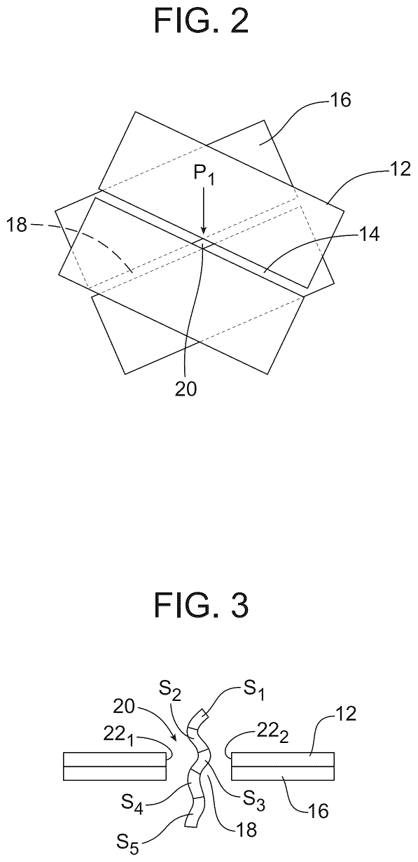

As illustrated in FIG. 2, when the first atomically-thin conducting layer 12 and first atomically-thin insulating layer 16 are stacked together a first nanopore 20 is formed at a first crossing point P₁ of the first nanogap 14 and the second nanogap 18 (i.1. the nanogaps cross at a non-zero angle). As should be appreciated, the first nanopore 20 extends through both of the layers 12, 16. The nanogaps 14, 18 are achieved by one-dimensional etching completely through the two dimensional layers 12, 16. The etch tracks or nanogaps 14, 18 may be very narrow (on the order of 10 nm or less) in width and may have a length greater than 100 nm and may be formed in parallel resulting in arrays. One possible approach for completing the etching of the nanogaps in the conducting layer 12 and the insulating layer 16 is disclosed in US 2020/0071817, the full disclosure of which is incorporated herein for reference.

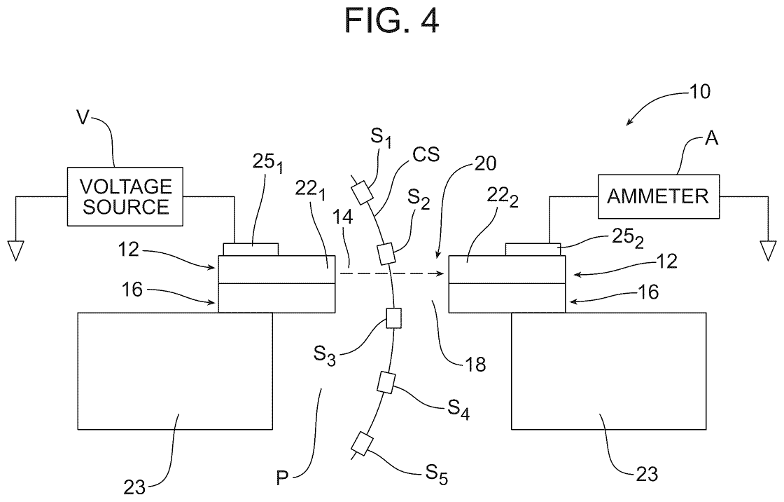

As illustrated in FIG. 3, two of the four edges are formed by physically separated conducting sheets and the first nanogap 14 may form a first electrode pair 22₁, 22₂. That electrode pair 22₁, 22₂ may be used to perform lateral electrical detection of molecules and particles/chemical substance CS passing through the nanopore 20. In the illustrated embodiment, the chemical substance CS is a complex chemical substance. Such a complex chemical substance CS may include, for example, (a) deoxyribonucleic acid (DNA) or ribonucleic acid (RNA) wherein the segments S₁-S₅ of the chemical substance represent different nucleic acids or (b) a protein wherein the segments S₁-S₅ represent different amino acids. Of course, it should be appreciated that the chemical substance CS to be detected or identified could be substantially any molecule, particle, ion, or element.

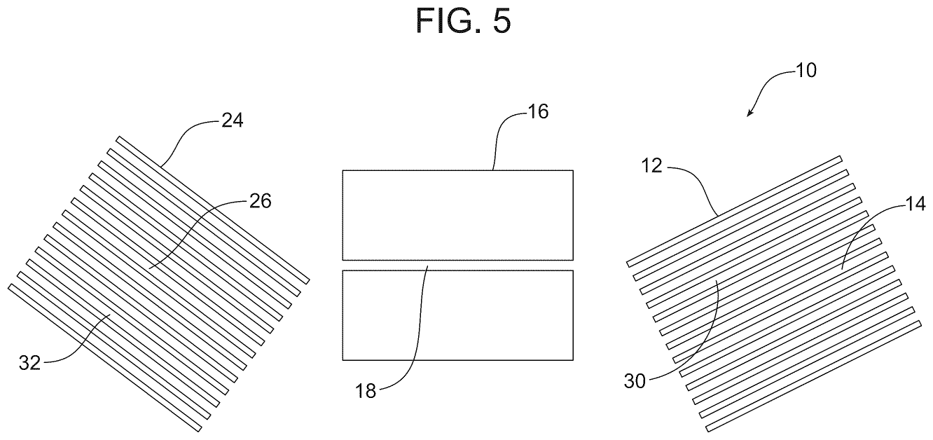

More particularly, as illustrated in FIG. 4, the heterostructure device 10 of FIG. 3 may be supported on a pore P in a porous support substrate or free-standing membrane 23 that may be made from any number of appropriate materials including, but not necessarily limited to, silicon, silicon dioxide (SᵢO₂), silicon nitride (SᵢNₓ), or the like.

Standard nano and micron lithography methods are then used to make electrical contacts 25₁, 25₂ to the two electrodes 22₁, 22₂ of the first conducting layer 12. The electrical contact 25₁ to the electrode 22₁ is connected to a voltage source V while the electrical contact 25₂ to the second electrode 22₂ is connected to a current (ammeter) meter A.

In operation, the voltage source V induces a sensing current (denoted by the dashed line across the nanopore 20) that passes through the molecule/chemical substance CS which is measured by the external ammeter A and distinguishes between the various building blocks of the molecules as it translocates through the nanopore 20.

Cross-gap-nanostructure devices 10 may be made with multiple conducting layers and/or multiple insulating layers that are stacked in an alternating pattern. Since alignment of etch tracks/nanogaps can be technically challenging, the conducting layers and/or insulating layers may be made with multiple etch tracks/nanogaps that increase the probability of aligning etch tracks/nanogaps in multiple stacked layers of conducting and insulating materials while also providing the possibility of the construction of an array of independently electronically addressable nanopores.

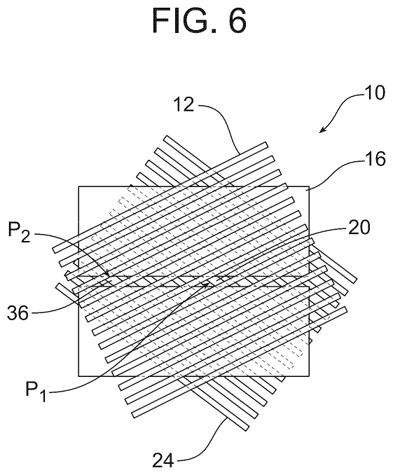

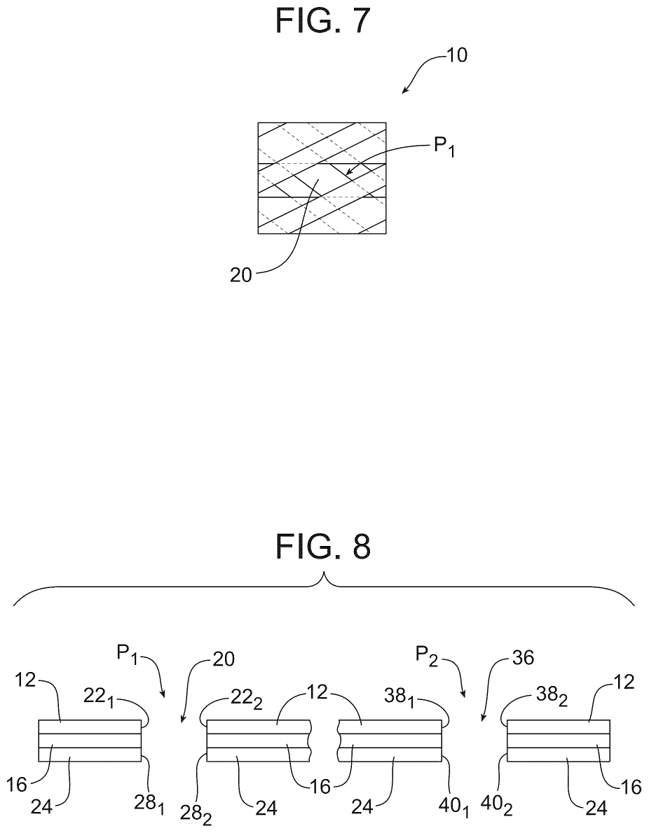

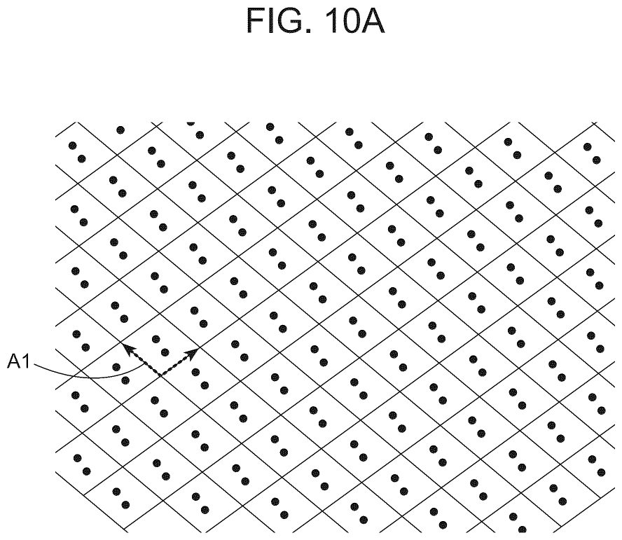

Reference is now made to FIGS. 5-8 illustrating a second possible embodiment of cross-gap-nanopore heterostructure device 10 including (a) a first atomically-thin conducting layer 12 having a plurality of nanogaps including a first nanogap 14, (b) a first atomically-thin insulating layer 16 having a second nanogap 18 and (c) a second atomically-thin conducting layer 24 having a plurality of nanogaps including a third nanogap 26. When the first atomically-thin conducting layer 12, first atomically-thin insulating layer 16, and second atomically-thin conducting layer 24 are stacked in alternating fashion, the first atomically-thin insulating layer 16 is sandwiched between the two atomically-thin conducting layers 12, 24 (see FIGS. 6 and 8).

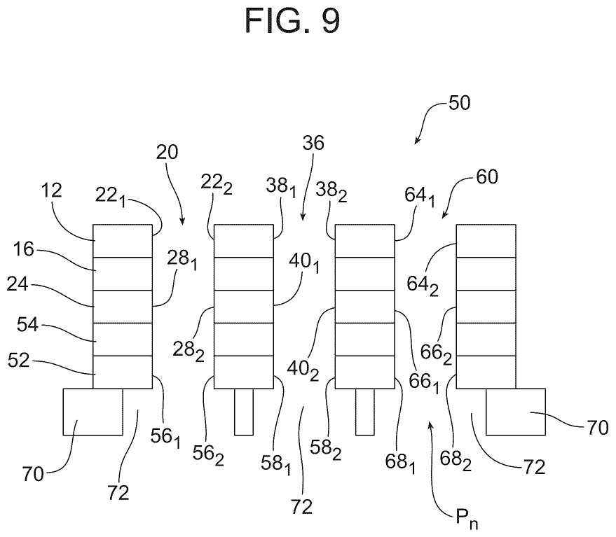

As shown in FIGS. 7 and 8, the third nanogap 26 crosses the first nanogap 14 and the second nanogap 18 at the first crossing point P₁ so that the first nanopore 20 also extends through the second atomically-thin conducting layer 24. The third nanogap 26 may form a second electrode pair 28₁, 28₂ within the first nanogap 20.

As illustrated in FIGS. 5 and 6, the first conducting layer 12 and the second conducting layer 24 may include additional nanogaps 30, 32, respectively, that cross the second nanogap 18 in the insulating layer 16 at a second crossing point P₂ forming a second nanopore 36.

The nanogap 30 in the first conducting layer 12 may form a third electrode pair 38₁, 38₂ within the second nanopore 36 while the second conducting layer may form a fourth electrode pair 40₁, 40₂ within the second nanopore.

The side view slice of the structure (FIG. 8) shows that we now have four electrically isolated electrodes 22₁, 22₂, 28₁, 28₂ that form the sidewalls of the nanopore 20 and four electrically isolated electrodes (38₁, 38₂) and (40₁, 40₂) that form the sidewalls of the nanopore 36. These four electrodes (22₁, 22₂), (28₁, 28₂) and (38₁, 38₂), (40₁, 40₂) allow for the ability to simultaneously control the voltage drops along the length of the nanopores 20, 36 and transversely to it (in the lateral direction), thus providing greater electrical control of its porosity. In addition, the two pairs of electrodes (22₁, 22₂), (28₁, 28₂) and (38₁, 38₂), (40₁, 40₂) now have the ability to simultaneously probe the electrical response of the pore contents at its bottom and top surfaces. This simultaneous electrical probing can permit time of flight detection of species as they move through the pore, while also permitting cross checking of measurements which is especially important for complex molecular detection (as in DNA sequencing).

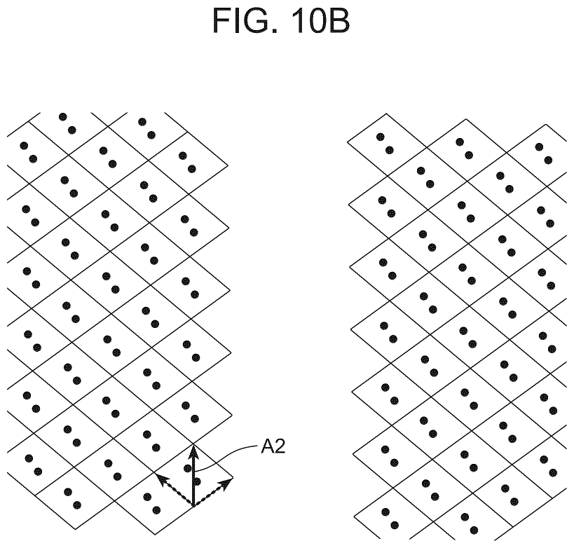

As illustrated in FIG. 9, the cross-gap-nanopore heterostructure device 50 may include any number of alternating additional atomically-thin conducting layers 52 and additional atomically-thin insulating layers 54 providing (a) additional electrode pairs 56₁, 56₂ in the first nanopore 20 and additional electrode pairs 58₁, 58₂ in the second nanopore 36, (b) additional nanopores 60 at additional crossing points Pₙ or (c) both additional electrode pairs in first and second nanopores and additional nanopores at additional crossing points. Those additional nanopores 60 may, in turn, include one or more additional electrode pairs (64₁, 64₂) in the first atomically-thin conducting layer 12, (66₁, 66₂) in the second atomically-thin conducting layer 24 and (68₁, 68₂) in the additional atomically-thin conducting layer 52.

As also illustrated in FIG. 9, the cross-gap-nanopore heterostructure device 50 may be stacked upon and supported by a porous support substrate 70 including a plurality of pores 72. Those pores 72 may be larger than the nanopores 20, 36, 60 and are typically microns or more. Where the pores 72 align with the nanopores 20, 36, 60, chemical substances may flow through the heterostructure device 50 and the porous support substrate 70.

The porous support substrate 70 may be made from any appropriate material including, but not necessarily limited to SiN_{X} membrane frame, SiO₂ and Si. Further, while not shown in FIGS. 1-8, the heterostructure devices 10, 10′ could also be stacked or supported upon such a substrate.

In all cases, the various etched 2D layers are stacked through various mechanical transfer schemes that have been developed (using, e.g., sacrificial polymer layers that maintain the etch track dimensions and structure). This stacking onto the substrate holes can be achieved either after the holes in the substrate are formed, or prior to their formation utilizing a selective etch that does not disturb the 2D multilayer pore structure of interest. Moreover, it is also possible that these structures can be obtained through 1D etching procedures performed after their placement (or growth) on a substrate.

The devices 10, 50, described above, include dimensions relevant to tunneling and electrostatic molecular sequencing. The devices 10, 50 include atomically-thin, crystallographically-ordered, and defect-free nanogap edges that form electrode pairs (22₁, 22₂), (28₁, 28₂), (38₁, 38₂), (40₁, 40₂), (64₁, 64₂), (66₁, 66₂) and (68₁, 68₂) for effectively probing a molecular sequence. Such devices 10, 50, based on tunneling, are shown to be superior over those based on electrostatics and gating for the application of molecular sequencing.

### Overview of Electrostatic Coupling and Gating Devices

Electrostatic sensing devices rely on the coulomb interaction with molecules and chemical species for detection signals. The coulomb interaction is the force between charges and is given by F=k_e (q_1 q_2)/r\hat{ }2, with k_e the coulomb constant, q_1 and q_2 the two charges, and r the distance between the charges. For the detection of the chemical and molecular species, the relevant coulomb force is approximately that given by individual charges with magnitude of the electron charge, q_e=1.6×10\hat{ }(−19) C. So the relevant force is

$$\begin{matrix}F = k\_e & q\_e \hat{} 2 / r \hat{} 2\end{matrix} \qquad ( 1 )$$

### Overview of Tunneling Devices

The tunneling devices 10, 50, disclosed in this document, function on the ability for charged particles to tunnel through classically forbidden regions and is described by an exponential probability for transmission through such a barrier. This transmission probability can be approximated by the well-known one-dimensional solution to the Schrödinger equation for the probability of tunneling into a barrier to a distance of r given by P=e\hat{ }(−2r√(2m/h\hat{ }2 (V−E))), with m being the mass of the particle, V−E being the difference in the potential energy and the energy of the particle, and h being the reduced planck's constant (1.1×10\hat{ }(−34) Js=6.6×10\hat{ }(−16) eVs). For tunneling from electronic materials, the mass of the electron m_e=9.1×10\hat{ }(−31) kg can be used, while the difference in energy is replaced with the work function Φ of the material (typically several eV for most materials). For the case of graphene, the work function is about Φ=4.3 eV. To parameterize changes in the tunneling, we can introduce a constant b=2√((2m_e)/h\hat{ }2Φ), which is about 2.1×10\hat{ }10/m. This results in a tunneling probability from graphene into a vacuum energy barrier of about

$$P = e \hat{} ( - br ) = e \hat{} ( - ( 2.1 \times 10 \hat{} 10 / m ) r ) \qquad ( 2 )$$

### Spatial and Detection Resolutions for Sequencing Molecules with Electrostatic/Gating Devices

The ability to sequence molecules requires that the fundamental underlying physics is able to selectively discern different monomers at molecular relevant displacements scales. The molecular relevant displacement scale (δr) is the atomic distance of about 0.3 nm, or 3.0×10\hat{ }(−10)m. This scale can be thought of as the monomer distance between adjacent base pairs in DNA (given approximately by 0.34 nm). This is also generally the rough scale (within a factor of about 2) that emerges for chemically bonded materials. For example, the level spacing between graphene sheets in few-layer graphene films is also approximately 0.34 nm. It is important to note that this length scale does not appreciably change for the detection of other polymer molecules, such as protein, so the arguments we provide are generally valid.

For a sequencing technology to be viable, the underlying physical phenomena (whether electrostatic coupling or quantum tunneling) must be highly selective to a specific monomer of the polymer chain. This selectivity means that the physical phenomena must vary by a large amount over the relevant displacement scale for sequencing (δr≈0.3 nm). Over this displacement scale, the electrostatic force will change by a magnitude of δF and the tunneling probability by a magnitude of δP. These changes can be approximated by δF≈|δr dF/dr| and δP≈|δr dP/dr|. We can make a direct comparison between the ability of these two phenomena to sequence by comparing their fractional changes δF/|F| and δP/|P|. These fractional changes must be large, being close to unity or even greater, for a good sequencing device.

For the case of electrostatic forces, the fractional change is

$$δ F / \vert F \vert \approx 2 δ r / r \qquad ( 3 )$$

This fractional change will increase as the distances r from the molecule to the electrode is decreased. To estimate the largest fractional change we could expect to obtain with an electrostatic device we can use the molecular displacement scale as the value for r, yielding an upper limit for δF/F≈2. While this could be a satisfactory physical process for sequencing, at this close approach to the electrode, the electrode uniformity would be required to be precise on the atomic (sub-nanometer) scale. As the molecule-electrode distance is increased, this fractional change will be significantly reduced. For example, at an electrode-molecule distance of 6.0 nm the fractional change is δF/F≈0.1. This fractional change means that adjacent monomers would only have a signal reduced in magnitude by about 10% in comparison to the monomer of interest. Thus, the device would likely be insufficient to pick out and identify a single monomer amongst the complete polymer chain.

### Spatial and Detection Resolutions for Sequencing Molecules with Tunneling Devices

In contrast to the limited spatial resolution and utility for sequencing found above for electrostatic interactions, the fractional change for tunneling devices is given by the constant large value of

$$δ P / \vert P \vert = b δ r \approx 6.3 \qquad ( 4 )$$

Unlike the electrostatic case, the fractional change for tunneling does not depend on the electrode-molecule distance, and is always larger than unity. Thus, tunneling should always be sufficient to spatially pick out and identify single monomers within a polymer chain, as long as the distances between the electrodes and the molecule are well defined, the electrode is atomically thin, and the arrangement provides a detectable tunneling current. These stringent constraints on the electrode properties are described in detail below.

### Device Constraints for Sequencing with Tunneling

The ability to utilize the selectivity of tunneling requires several stringent size constraints to the electrode systems. First, the uniformity of the electrode must be within δr in order to provide a uniform distance for tunneling to the molecule. This requirement essentially means that the electrode must be crystallographically uniform. In addition to being crystallographically uniform, the thickness of the electrode will also need to be within δr, otherwise more than one monomer would be measured simultaneously.

The last constraint is that the tunneling current must be sufficient in order for the measurement apparatus to detect it. Typically, the measurement apparatus will have a threshold current measurement sensitivity I_meas\hat{ }*. The 2-dimensional current density on the short length (ballistic) scale is given by j_2D=q_en_2D v_F, with the free charge carrier density in the two dimensional sheet given by n_2D (≈10\hat{ }17/m\hat{ }2 in graphene), and the Fermi velocity of carriers given by v_F (≈3×10\hat{ }6 m/s in graphene). To obtain a current to the molecule from the electrode we can approximate the width of the device to be δr and the probability for each electron to transmit to the molecule given by the tunneling probability P. This gives for the current I=q_e n_2D v_F δrP, which must be greater than I_meas\hat{ }* for it to be detectable. This results in the condition for detectability

$$e \hat{} ( - br ) \geq I\_meas {\hat{}}^{*} / ( ( {q\_en}_{-} 2 Dv\_Fδ r ) \qquad ( 5 )$$

$$or$$

$$r \leq ( - ln [ ( I\_meas {\hat{}}^{*} ) / ( ( {q\_en}_{-} 2 Dv\_Fδ r ) ] ) / b \qquad ( 6 )$$

For a threshold current of fempto-amps (10\hat{ }(−15) amps) the above condition requires an electrode distance of about 1.1 nm or less. Since the fractional tunneling probability is considerably greater than 1, it could be possible to reduce the value of b while still maintaining the spatial selection of a single monomer by altering the local environment (such as the liquid in which the polymer is suspended within). Altering the local environment in this way could alter the energy height of the tunneling barrier (essentially altering the work function of the graphene) and, as a result, extend the workable distance between the electrode and the molecule up to about 9 nm. An additional nanometer of workable distance might be obtainable by image charge correction in the vicinity of the electrodes, bringing the overall working distance to about 10 nm. It is important to emphasize that the uniformity of the edge of the graphene electrode would need to be maintained at the δr≈0.3 nm scale so that the overall distance to the molecule would be maintained.

### Further Considerations for Tunneling Device Dimensions

In the above discussion, we have considered only a single tunneling distance. In actuality, there would be two tunneling steps, one to tunnel to the monomer from the initial electrode followed by a second tunneling process to the other electrode. Since the tunneling represents a probability for overall transmission from the first to the second graphene electrode, the total probability can be formed as a product of probabilities (neglecting the special cases of strong interference effects). That is

$$P\_total = ( P\_ 1 ) ( P\_ 2 ) = e \hat{} ( - br\_ 1 ) e \hat{} ( - br\_ 2 ) = e \hat{} ( - b ( r\_ 1 + r\_ 2 ) ) = e \hat{} ( - bW ) \qquad ( 7 )$$

with W being the fixed width (electrode-to-electrode distance) of the nanogap subtracting off the size extent of the molecule within the gap between the electrodes. Thus, the tunneling should not be strongly altered by the exact location of the monomer within the nanogap, as long as the nanogap were uniform on the atomic scale. The devices 10, 50 described in this document, meet these requirements.

As a result, the cross-gap-nanopore heterostructure devices 10, 50 are useful in a method of determining a chemical substance CS. That method may be broadly described as including the step of passing the chemical substance CS through at least one nanopore 20, 36, 60 in a cross-gap-nanopore heterostructure 10, 50.

The passage of the chemical substance CS through the nanopore 20 (known as the “translocation process”) can be achieved in several ways as listed below:
1. Let the molecules diffuse through the nanopore. This is essentially a random process, using naturally occurring random fluctuations in the environment to push the molecules through.
2. Pumping a solution through the membrane to drive the molecules through.
3. Using surface energy gradients to drive the molecules through the membrane. By placing a drop of liquid with the molecules on one side of the membrane, the tendency for the drop to spread to the other side of the nanopore will drive molecules through the pore.
4. Using a density gradient of molecules across the nanopore will tend to yield the translocation of molecules from the higher density side to the lower density side.
5. Using “external electrodes” to drive ions contained in solution through the nanopore that will tend to subsequently drive the molecule of interest through the pore.
6. Using “external electrodes” to directly drive the molecule of interest (the one to be detected) through the nanopore. The driving mechanism can be through the molecule being charged or through its polarizability (i.e., a dielectrophoretic effect).
7. The integrated electrode pairs to the cross-gap nanopores can have a constant or alternating voltage applied between them that will tend to pull molecules of interest into the nanopore.

The method may include performing lateral electrical detection of the chemical substance CS as the chemical substance passes through the at least one nanopore 20, 36, 60. This is accomplished by applying a voltage difference to a first electrode pair 22₁, 22₂ in the at least one nanopore 20. In some embodiments, the method includes applying voltage differences to a plurality of different electrode pairs (22₁, 22₂), (28₁, 28₂), (38₁, 38₂), (40₁, 40₂), (64₁, 64₂), (66₁, 66₂) and (68₁, 68₂) in at least one nanopore 20, 36, 60.

The method may include the step of simultaneously electrically probing the chemical substance CS with the plurality of different electrode pairs (22₁, 22₂), (28₁, 28₂), (38₁, 38₂), (40₁, 40₂), (64₁, 64₂), (66₁, 66₂) and (68₁, 68₂) in the at least one nanopore 20, 36, 60 as the chemical substance passes through the at least one nanopore.

Still further, the method may include the step of influencing the flow of the chemical substance CS through the at least one nanopore 20, 36, 60 by application of dielectrophoretic forces.

Numerous benefits and advantages are provided by the cross-gap-nanopore heterostructure devices 10, 50 disclosed herein. The provision of multiple electrode pairs within a single nanopore allows for the ability to simultaneously control the voltage drops along the length of the nanopore and transversely to it (in the lateral direction)—thus providing greater electrical control of its porosity. In addition, the multiple pairs of electrodes now have the ability to simultaneously probe the electrical response of the pore contents at its bottom and top surfaces as well as multiple points in between. This allows for the transverse simultaneous current detection (in the lateral direction) of different portions of the same molecule as it translocates through the pore. This simultaneous electrical probing can permit time of flight detection of species as they move through the pore, while also permitting cross checking of measurements—especially important for complex molecular detection (as in DNA and RNA sequencing).

One of the important advantages of the multiple electrode pair formulation of cross-gapped nanopores is that it permits much greater accuracy in the sequencing determination. First, the second pair allows for a greater number of measurements of the same molecule which helps to significantly reduce read errors. Second, this configuration allows for the determination of changes of molecular directional switches to allow for the correct real-time sequencing. Using only one of the electrode pairs presents challenges in detecting the correct sequence. With the second electrode pair measurements, the change from leading to lagging gives unambiguous real-time determination of the translocation directional change that also allows for correct sequencing. By using additional electrode stacks, even greater improvements to the accuracy of the sequencing measurements can be achieved.

The foregoing has been presented for purposes of illustration and description. It is not intended to be exhaustive or to limit the embodiments to the precise form disclosed. Obvious modifications and variations are possible in light of the above teachings. For example, while the insulating layers 16, 54 in the above-described embodiments include only a single etch track/nanogap 18, it should be appreciated that the insulating layers may also include an array of etch tracks/nanogaps to increase the probability of nanopore formation by alignment of those etch tracks/nanogaps with etch tracks/nanogaps of the other conducting and insulating layers of the heterostructure.

The cross-gap-nanopore heterostructures may also consist of the following alternative combinations. The cross-gap-nanopore heterostructures may consist only of conducting layers, only of insulating layers and only of semiconducting layers. The cross-gap-nanopore heterostructures may also not permit electrode pairs. Such novel nanopores could still provide detection and sequencing of molecular species having different properties and dimensions.

All such modifications and variations are within the scope of the appended claims when interpreted in accordance with the breadth to which they are fairly, legally and equitably entitled.

What is claimed:

1. A heterostructure device, comprising:
a first conducting layer including a first nanogap having a width of 10 nm or less, a length of greater than 100 nm and a depth extending through the first conducting layer;
a first insulating layer including a second nanogap having a width of 10 nm or less, a length of greater than 100 nm and a depth extending through the first insulating layer; and
a first nanopore formed at a first crossing point of the first nanogap and the second nanogap wherein the first nanopore extends through the first conducting layer and the first insulating layer.

2. The heterostructure device of claim 1, wherein the first conducting layer is atomically thin having a thickness of about 1.0 nm or less.

3. The heterostructure device of claim 2, wherein the first insulating layer is atomically thin having a thickness of about 1 nm or less.

4. The heterostructure device of claim 3, wherein the first nanogap forms a first electrode pair.

5. The heterostructure device of claim 1, wherein the first nanogap and the second nanogap are etched and include edges that are crystallographically-ordered and parallel on an atomic scale.

6. The heterostructure of claim 3, further including a second atomically-thin conducting layer including a third nanogap, wherein (a) the first atomically-thin insulating layer is sandwiched between the first atomically-thin conducting layer and the second atomically-thin conducting layer and (b) the third nanogap crosses the first nanogap and the second nanogap at the first crossing point so that the first nanopore also extends through the second atomically-thin conducting layer.

7. The heterostructure of claim 6, wherein the third nanogap includes edges that are crystallographically-ordered and parallel on an atomic scale.

8. The heterostructure of claim 7, wherein the first nanogap forms a first electrode pair within the first nanopore and the second nanogap forms a second electrode pair within the first nanopore.

9. The heterostructure of claim 8, wherein the first conducting layer and the second conducting layer include additional nanogaps that cross the second nanogap in the atomically-thin insulating layer at a second crossing point forming a second nanopore.

10. The heterostructure of claim 9, wherein the additional nanogaps in the first conducting layer and the second conducting layer form additional electrode pairs within the second nanopore.

11. The heterostructure of claim 9 further including alternating additional atomically-thin conducting layers and additional atomically-thin insulating layers providing (a) additional electrode pairs in the first nanopore and the second nanopore, (b) additional nanopores at additional crossing points or (c) additional electrode pairs in the first nanopore and the second nanopore and additional nanopores at additional crossing points.

12. The heterostructure of claim 11, wherein the first atomically-thin conducting layer, the second atomically-thin conducting layer and the additional atomically-thin conducting layers are made from a material selected from a group consisting of graphene, transition metal dichalcogenides (TMDs), borophene, germanene, silicene, stanene, plumbene, phosphorene, antimonene, Si₂BN, borocarbonitrides and combinations thereof.

13. The heterostructure of claim 12, wherein the first atomically-thin insulating layer and the additional atomically-thin insulating layers are made from a material selected from a group consisting of hexagonal boron nitride, transition metal dichalcogenides (TMDs), bismuthene, borocarbonitrides and combinations thereof.

14. A cross-gap-nanopore heterostructure, comprising: (a) a first conducting layer including a first nanogap having a width of 10 nm or less, a length of greater than 100 nm and a depth extending through the first conducting layer, (b) a first insulating layer including a second nanogap having a width of 10 nm or less, a length of greater than 100 nm and a depth extending through the first insulating layer, and (c) a first nanopore formed at a first crossing point of the first nanogap and the second nanogap wherein (a) the first nanopore extends through the first conducting layer and the first insulating layer, (b) the first nanopore and the second naopore are etched and include edges that are crystallographically-ordered and parallel on an atomic scale, and (c) the cross-gap-nanopore heterostructure is adapted for real-time determination of nucleotide sequencing of a strand of genetic material.

15. The cross-gap-nanopore heterostructure of claim 14, wherein the genetic material is selected from a group consisting of RNA, DNA and combinations thereof.

16. The cross-gap-nanopore heterostructure of claim 14, including (a) a plurality of alternating atomically-thin conducting layers and insulating layers and (b) at least one nanopore having stacked electrode pairs.

17. The cross-gap-nanopore heterostructure of claim 14, including (a) a plurality of alternating atomically-thin conducting layers and insulating layers and (b) a plurality of nanopores having at least one individually addressable electrode pair.

18. A method of determining a chemical substance, comprising:
passing the chemical substance through at least one nanopore in a cross-gap-nanopore heterostructure.

19. The method of claim 18, including performing lateral electrical detection of the chemical substance as the chemical substance passes through the at least one nanopore.

20. The method of claim 19, including (a) simultaneously electrically probing the chemical substance with a plurality of different electrode pairs in the at least one nanopore as the chemical substance passes through the at least one nanopore and (b) influencing flow of the chemical substance through the at least one nanopore through application of dielectrophoretic forces.
